(19) United States
(12) Patent Application Publication
(10) Pub. No.: US 2016/0039726 A1
(43) Pub. Date: Feb. 11, 2016

(54) Boroaluminosilicate Molecular Sieves and Methods for Using Same for Xylene Isomerization

(71) Applicant: Jeffrey Allen AMELSE, (US)

(72) Inventor: Jeffrey A. Amelse, Batavia, IL (US)

(73) Assignee: BP Corporation North America Inc., Naperville, IL (US)

(21) Appl. No.: 14/777,025

(22) PCT Filed: Mar. 12, 2014
(86) PCT No.: PCT/US14/24421

§ 371 Date: Sep. 15, 2015

Related U.S. Application Data
(60) Provisional application No. 61/793,180, filed on Mar. 15, 2013.

(51) Int. Cl. C07C 5/27; B01J 29/86; B01J 35/02; B01J 8/04

(52) U.S. Cl. CPC C07C 5/2737; B01J 8/04; B01J 29/86; B01J 35/023; C07C 5/2775; B01J 2208/027; C07C 2529/86

(57) ABSTRACT

Boroaluminosilicate molecular sieve catalysts are provided and are useful for hydrocarbon conversion reactions including isomerization of xylenes in C8 aromatics feedstocks to produce p-xylene. Advantageously, it has been found that the boroaluminosilicate molecular sieve catalysts of the invention are more selective than conventional commercial xylene isomerization catalysts, resulting in reduced formation of transmethylation byproducts (C7 and C9 aromatics) while simultaneously providing a high degree of xylene isomerization.

### FIELD OF THE INVENTION

The disclosure relates to methods for making and using an isomerization catalyst, and in particular, methods for making and using boroaluminosilicate molecular sieves, and catalyst systems and isomerization reactors containing the same in xylene isomerization.

### BACKGROUND

Xylene isomerization is an important chemical process. P-xylene is useful in the manufacture of terephthalic acid which is an intermediate in the manufacture of polyesters. Typically p-xylene is derived from mixtures of C₈ aromatics separated from such raw materials as petroleum reformates, usually by distillation. The C₈ aromatics in such mixtures are ethylbenzene, p-xylene, m-xylene, and o-xylene.

Xylene isomerization catalysts can be classified into three types based upon the manner in which they convert ethylbenzene: (1) naphthene pool catalysts, (2) transalkylation catalysts, and (3) hydrodeethylation catalyst. Naphthene pool catalysts, containing a strong hydrogenation function (e.g, platinum) and an acid function (e.g., a molecular sieve) can convert a portion of the ethylbenzene to xylenes via naphthene intermediates. Transalkylation catalysts generally contain a shape selective molecular sieve which inhibits certain reactions based on the size of the reactants, products, and/or intermediates involved. For example, the pores can allow ethyl transfer to occur via a dealkylation/realkylation mechanism, but can inhibit methyl transfer which requires the formation of a bulky biphenylalkane intermediate. Finally, hydrodeethylation catalysts, containing an acidic shape-selective catalyst and an ethylene-selective hydrogenation catalyst, can convert ethylbenzene to benzene and ethane via an ethylene intermediate. However, such catalysts often sacrifice xylene isomerization efficiency to efficiently remove ethylbenzene.

In contrast, dual bed catalyst systems can more efficiently convert ethylbenzene and non-aromatics in mixed C₈ aromatic feeds, while simultaneously converting xylenes to thermal equilibrium with a distribution of the xylene isomers (paraxylene:metaxylene:orthoxylene) of approximately 1:2:1. Dual bed xylene isomerization catalysts consist of an ethylbenzene conversion catalyst component and a xylene isomerization component. Typically, the ethylbenzene conversion catalyst is selective for converting ethylbenzene to products which can be separated via distillation, but it is less effective as a xylene isomerization catalyst; that is, it does not produce an equilibrium distribution of xylene isomers. A dual bed catalyst system has an advantage over a conventional single bed xylene isomerization catalyst in that it affords lower xylene losses. However, in order to maximize p-xylene yields from dual bed catalyst systems, the xylene isomerization component should demonstrate high xylene isomerization activity, but low xylene loss to prevent degradation of catalytic selectivity.

Borosilicate molecular sieves have been employed commercially for hydrocarbon conversion reactions including isomerization of xylenes in C₈ aromatics to produce p-xylene. Catalyst compositions, generally useful for hydrocarbon conversion, based upon AMS-1B crystalline borosilicate molecular sieve have been described in U.S. Pat. Nos. 4,268,420; 4,269,813; 4,285,919; and Published European Application No. 68,796. The catalyst compositions typically are formed by incorporating an AMS-1B crystalline borosilicate molecular sieve material into a matrix such as alumina, silica or silica-alumina to produce a catalyst formulation. Borosilicate sieves have low intrinsic catalytic activity and typically must be used in conjunction with an alumina support to impart activity.

Sulikowski et al. in Z. Phys. Chem., 177, 93-103 (1992) examined the catalytic isomerization of xylenes at 300° C. and 445° C. on zeolite [Si, B, Al]-ZSM-5 (an MFI boroaluminosilicate molecular sieve) prepared under non-alkaline conditions (without the addition of a base). The boroaluminosilicate molecular sieves were prepared via a novel synthetic route where fluoride ions were used to solubilize the silicon and aluminum in the synthesis gel, and were produced with large particle sizes (on the order of tens to hundreds of microns in size). These authors and others (See for example J. Wei, J. Catal., 76, 433 (1982), and J. Amelse, Proc. 9th International Zeolite Conf., Montreal, 1992, Eds. R. von Ballmoos, et al., Butterworth-Heinemann, p. 457 (1993)), note that xylene isomerization over MFI zeolites is diffusion limited with pX having a higher diffusion rate than that of o-xylene and m-xylene. The data provided in FIG. 2 of the Sulikowski article do not show a % pX/(% pX+% mX+% oX) of greater than 20% and the data of FIG. 3 exhibit a p-xylene content after isomerization of just over 20% compared to an equilibrium value of about 24%. Thus, the large particle boroaluminosilicate molecular sieves prepared by Sulikowski et al. would not be ideal for the xylene isomerization catalyst of a dual bed catalyst system. The Sulikowski article also notes that it has been experimentally confirmed that aluminum free [Si, B]-ZSM-5 is totally inactive for the isomerization of xylenes.

Thus, there continues to be a need for improved xylene isomerization catalysts that can maximize yields of p-xylene while minimizing xylene loss to transmethylation reactions. In particular there continues to be a need for small particle molecular sieves that have low diffusion resistance and high activity for the isomerization of xylenes while minimizing xylene loss to transmethylation reactions.

### BRIEF SUMMARY OF THE INVENTION

The present invention provides boroaluminosilicate molecular sieves for use as xylene isomerization catalysts. Such boroaluminosilicate molecular sieves have surprisingly been found to exhibit unexpectedly high xylene isomerization activity while simultaneously yielding less transmethylation byproducts (C₇ and C₉ aromatics) compared to industry standard catalysts. Also provided are methods for use of these boroaluminosilicate molecular sieves for enriching the p-xylene content of a hydrocarbon-containing feed stream comprising xylene isomers. Such catalysts include boroaluminosilicate molecular sieves that can be prepared, for example, in substantially H⁺-form through the use of an organic base, eliminating the need for a cation exchange step to remove alkali metal which can degrade isomerization performance.

Accordingly, in one aspect, the invention provides the hydrogen form of boroaluminosilicate molecular sieves having an average crystallite size less than 2 μm.

In another aspect, the invention provides methods for increasing the proportion of p-xylene (pX) in a hydrocarbon-containing feed stream comprising xylene isomers, said method comprising contacting the hydrocarbon-containing feed stream with an isomerization catalyst under conditions suitable to yield a stream enriched in p-xylene with respect to the hydrocarbon-containing feed stream, wherein the isomerization catalyst comprises a boroaluminosilicate molecular sieve prepared using an amine base.

In another aspect, the invention provides catalyst systems for enriching a xylene isomers feed in p-xylene comprising a first bed comprising an ethylbenzene (EB) conversion catalyst and a second bed comprising an isomerization catalyst that comprises a boroaluminosilicate molecular sieve.

In another aspect, the invention provides a xylene isomerization reactor having a reaction zone containing a catalyst system as described above.

### BRIEF DESCRIPTION OF THE DRAWINGS

FIG. 1a is a flow diagram illustrating one illustrative embodiment of a method for xylene isomerization.

FIG. 1b is a flow diagram illustrating another illustrative embodiment of a method for xylene isomerization.

FIG. 1c is a flow diagram illustrating a third illustrative embodiment of a method for xylene isomerization.

FIG. 2 shows SEM images of boroaluminosilicate molecular sieves prepared from using ethylenediamine as a base; (TOP) 0.34 wt % Al, 0.93 wt % B, 100% crystalline; (BOTTOM) 0.35 wt % Al, 0.66 wt % B, 97% crystalline.

FIG. 3 is a plot of net yield of toluene vs. % pX/xylenes (30-52% EB conversion data) for various molecular sieve catalysts.

FIG. 4 a plot of net yield of trimethylbenzene vs. % pX/xylenes for various molecular sieve catalysts.

FIG. 5 is a plot of net yield pX/net yield (toluene+trimethylbenzene) vs. pX/xylenes for various molecular sieve catalysts.

FIG. 6 is a plot of net yield of trimethylbenzenes vs. % pX/xylenes for various xylene isomerization catalysts tested according to Example 5 (infra).

### DETAILED DESCRIPTION OF THE PREFERRED EMBODIMENTS

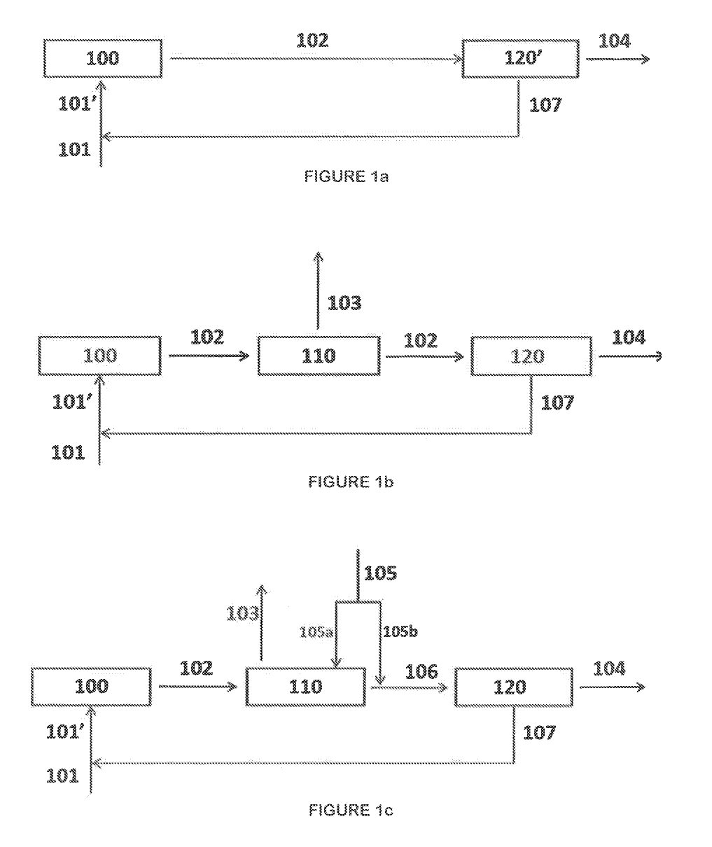

In a first aspect, the invention provides methods for increasing the proportion of p-xylene (pX) in a hydrocarbon-containing feed stream including xylene isomers. The method includes, referring to FIG. 1a, contacting in a reaction zone of a reactor (100) a hydrocarbon-containing feed stream (101 or 101′) with an isomerization catalyst of the application under conditions suitable to yield a stream enriched in p-xylene (102) with respect to the hydrocarbon-containing feed stream, where the isomerization catalyst includes a boroaluminosilicate molecular sieve. The pX enriched stream (102), can generally contain benzene, toluene, and xylene isomers (i.e., ethylbenzene (EB), o-xylene (oX), m-xylene (mX) and p-xylene (pX)). The methods may be carried out as batch, semi-continuous, or continuous operations.

In certain embodiments, the hydrocarbon-containing feed stream includes at least 80 wt. % xylene isomers and a pX/X of less than 12 wt. %. The term “pX/X” refers to the weight percent of p-xylene (pX) in a referenced stream or product with respect to the total xylenes in the same stream or product (i.e., the sum of o-xylene, m-xylene, and p-xylene).

Suitable conditions for contacting the hydrocarbon-containing feed stream with the isomerization catalyst include liquid, vapor, or gaseous (supercritical) phase conditions in the presence or substantial absence of hydrogen. In certain embodiments, the hydrocarbon-containing feed stream is contacted with the isomerization catalyst in the presence of hydrogen. In certain other embodiments, the hydrocarbon-containing feed stream is contacted with the isomerization catalyst in the absence of hydrogen.

Typical vapor phase reaction conditions include a temperature of from about 500° F. to about 1000° F. In certain embodiments, the temperature is from about 600° F. to about 850° F. In certain embodiments, the temperature is from about 700° F. to about 800° F.

Typical vapor phase reaction pressure can be from about 0 psig to about 500 psig. In certain embodiments, the pressure can be from about 100 to about 300 psig.

Typical vapor phase reaction may also include an H₂/hydrocarbon mole ratio of from about 0 to 10. In certain embodiments, the H₂/hydrocarbon mole ratio is from about 0.5 to about 4.

Typical vapor phase reaction may also include a liquid weight hourly space velocity (LWHSV) of hydrocarbon-containing feed stream from about 1 to about 100. In certain embodiments, the LWHSV is from about 4 to about 15.

For example, in one embodiment the pressure is from about 0 psig to about 500 psig, the H₂/hydrocarbon mole ratio is from about 0 to about 10, and the liquid weight hourly space velocity (LWHSV) is from about 1 to about 100. In certain embodiments, vapor phase reaction conditions for xylene isomerization include a temperature of from about 600° F. to about 850° F., a pressure of from about 100 to about 300 psig, an H₂/hydrocarbon mole ratio of from about 0.5 to about 4, and a LWHSV of from about 4 to about 15. Other typical vapor phase conditions for xylene isomerization are further described, for example, in U.S. Pat. No. 4,327,236.

Typical liquid phase conditions for xylene isomerization are described, for example, in U.S. Pat. No. 4,962,258. The liquid phase process temperature can be from about 350° F. to about 650° F., or from about 500° F. to about 650° F.; or from about 550° F. to about 650° F. The upper temperature of the range is chosen so that the hydrocarbon feed to the process will remain in the liquid phase. The lower temperature limit can be dependent on the activity of the catalyst composition and may vary depending on the particular catalyst composition used. The total pressure used in the liquid phase process should be high enough to maintain the hydrocarbon feed to the reactor in the liquid phase, but there is no upper limit for the total pressure useful in the process. In certain embodiments, the total pressure is in the range of about 400 psig to about 800 psig. The process weight hourly space velocity (WHSV) is typically in the range of about 1 to about 60 hr⁻¹; or from about 1 to about 40 hr⁻¹; or from about 1 to about 12 hr⁻¹. Hydrogen may be used in the process, up to the level at which it is soluble in the feed; however, in certain embodiments, hydrogen is not used within the process. In another embodiment hydrogen is added above solubility but the bulk of the hydrocarbons remain in a liquid phase, for example in a trickle bed reactor.

Typical conditions for xylene isomerization at supercritical temperature and pressure conditions are described, for example, in U.S. Pat. No. 5,030,788. Generally, supercritical conditions contact the isomerization catalyst at a temperature and pressure above the critical temperature and pressure of the mixture of components in said stream. For a typical hydrocarbon-containing feed stream including xylene isomers, the critical pressure is above about 500 psig and the critical temperature is above about 650° F. Hydrogen may optionally be added to the reactor feed stream, as a small amount of hydrogen may reduce the rate of catalyst deactivation. If hydrogen is added, it can be added at a level below its solubility in the isomerization stream at reactor pressure and at temperatures present in a feed-effluent heat exchanger to avoid the formation of a vapor phase and its associated low heat transfer coefficient.

The boroaluminosilicate molecular sieves can be prepared by, first, combining a boron source, an aluminum source, a silica sol, a template, and a base to form a reaction mixture.

The boron source may be any familiar to one skilled in the art for preparing molecular sieves, including for example boric acid. The silica sol can be commercially available colloidal silicas, for example, Ludox® HS-40 (40 wt. % suspension of colloidal silica in H₂O), Ludox® AS-40 (40 wt. % suspension of colloidal silica in H₂O, stabilized by ammonium hydroxide), and Nalco 2327, among others. NALCO 2327 has a mean particle size of 20 nm and a silica content of approximately 40 percent by weight in water with a pH of approximately 9.3, and ammonium as the stabilizing ion. Methods of making colloidal silica particles include, for example, partial neutralization of an alkali-silicate solution.

The aluminum source can be sodium aluminate, or can be alkali free, such as aluminum sulfate, aluminum nitrate, an aluminum C₁₋₁₀alkanoate, or an aluminum C₁₋₁₀alkoxide such as aluminum isopropoxide. The template may be any familiar to one skilled in the art for preparing molecular sieves, including for example tetra(C₁₋₁₀alkyl)ammonium compounds, such as tetra(C₁₋₁₀alkyl)ammonium hydroxide (e.g., tetra(propyl)ammonium hydroxide) or a tetra(C₁₋₁₀alkyl)ammonium halide (e.g., tetra(propyl)ammonium bromide).

The base can be either a Brønsted or Lewis base that, when dissolved in water, yields a basic solution (i.e., pH>7). That is, the present invention excludes boroaluminosilicate molecular sieves prepared using ammonium fluoride to facilitate the reactions forming the molecular sieves. In certain embodiments, the base is an alkali metal base or an alkaline earth metal base, such as, for example NaOH, KOH, Ca(OH)₂, and the like. In certain other embodiments, the base is an essentially metal-free base, such as, for example, ammonium hydroxide.

In certain other embodiments, the base is an amine base. The phrase “amine base,” includes (a) compounds containing at least one functional group (e.g., 1, 2, 3, 4 or more) of the formula, —NR₂, where each R is independently a hydrogen or C₁₋₄ alkyl, such as compounds of the formula R¹—NR₂, where R¹ is phenyl, naphthyl, pyridyl, quinolinyl, or C₁₋₁₀alkyl; and R₂N—R²—NR₂, where R² is phenyl, naphthyl, pyridyl, quinolinyl, or C₁₋₁₀alkyl; and (b) 5-10 membered heterocyclic (monocyclic or fused bicyclic aromatic, or monocyclic, fused bicyclic, or bridged bicyclic non-aromatic) compounds whose annular atoms include carbon, at least one optionally substituted annular nitrogen atom (e.g, 1, 2, or 3 annular nitrogens), and optionally one heteroatom selected from O and S. Examples of amine bases include, for example, aniline, 4-dimethylaminopyridine, pyridine, pyrazine, pyrimidine, triazine, tetrazine, quinoline, isoquinoline, imidazole, pyrazole, triazole, tetrazole, n-propylamine, n-butylamine, 1,2-ethylenediamine, 1,3-propylenediamine, 1,4-butylenediamine, N,N,N′,N′-tetramethyl-1,2-ethylenediamine, triethylamine, diisopropylethylamine, diisopropylamine, t-butyamine, iso-propylamine, pyrrole, N-methylpyrrole, pyrroline, pyrrolidine, imidazoline, imidazolidine, pyrazoline, pyrazolidine, N-methylpyrrolidine, piperidine, piperazine, morpholine, N-methylpiperidine, and mixtures thereof.

The term “alkyl,” means a straight or branched chain saturated hydrocarbon containing from 1 to 10 carbon atoms, unless otherwise specified. Representative examples of alkyl include, for example, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, for example, —CH₂—, —CH₂CH₂—, —CH₂CH₂CHC(CH₃), —CH₂CH(CH₂CH₃)CH₂—.

In one embodiment, the amine base comprises an C₁₋₁₀ alkylamine or a C₁₋₁₀ alkyldiamine. The term “alkylamine,” means an alkyl group, as defined above, substituted with one group of the formula —NR₂, where each R is independently a hydrogen or C₁₋₄ alkyl. The term “alkyldiamine,” means an alkyl group, as defined above, substituted with two groups of the formula —NR₂, where each R is independently hydrogen or C₁₋₄ alkyl, where the two —NR₂ groups are not attached to the same carbon atom.

In another embodiment, the amine base comprises an C₁₋₁₀ alkylamine (e.g., n-propylamine). In another embodiment, the amine base comprises a C₁₋₁₀ alkyldiamine (e.g., ethylenediamine). In certain embodiments of any of the preceding embodiments, the amine base is substantially-free of alkali metal cation, e.g., Na⁺.

The reaction mixture is warmed to provide a product mixture containing a solid. Suitably, the reaction mixture can be warmed to a temperature between 100° C. and 200° C.; or to a temperature between 150° C. and 170° C., for a suitable time to provide the product mixture containing the solid. For example, the reaction mixture can be heated to a suitable temperature in an autoclave at autogenous pressure. The solid is isolated from the product mixture, for example, by filtration or centrifugation.

Where the boroaluminosilicate molecular sieve is prepared using a base that contains alkali metal cations (e.g., Na⁺) and/or alkali earth cations (e.g., Mg²⁺), and/or using an alkali metal containing aluminum source (e.g., sodium aluminate), and/or using a silica sol stabilized by an alkali metal source, the solid can be contacted with a cation exchange solution containing an ammonium salt, such as ammonium acetate, in an amount and for a period of time suitable to exchange the alkali metal cations and/or alkali earth cations for hydrogen (i.e., to provide the H⁺-form of the boroaluminosilicate molecular sieve). However, the use of an amine base, as defined above, for the preparation of the boroaluminosilicate molecular sieve can avoid the necessity of cation exchange.

Ultimately, the resulting solid, with or without cation exchange, can be calcined to yield the boroaluminosilicate molecular sieve. The calcining is typically at a temperature between 480° C. and 600° C.

The boroaluminosilicate molecular sieves prepared according to the preceding methods typically have an MFI framework and can have an alkali metal content less than 400 ppmw (e.g., between about 10 ppmw and about 400 ppmw). In certain embodiments, the boroaluminosilicate molecular sieve has an alkali metal content is less than 350 ppmw (e.g., between about 10 ppmw and about 350 ppmw); or less than 300 ppmw (e.g., between about 10 ppmw and about 300 ppmw); or less than 250 ppmw (e.g., between about 10 ppmw and about 250 ppmw); or less than 200 ppmw (e.g., between about 10 ppmw and about 200 ppmw); or less than 150 ppmw (e.g., between about 10 ppmw and about 150 ppmw). In certain other embodiments, the boroaluminosilicate molecular sieve has an alkali metal content of less than 100 ppmw (e.g., between about 10 ppmw and about 110 ppmw).

The boron content of the boroaluminosilicate molecular sieves prepared as above can range from about 0.01 wt. % to about 1.5 wt. %. In certain embodiments, the boron content ranges from about 0.01 wt. % to about 1.2 wt. %; or about 0.01 wt. % to about 1.0 wt. %; or about 0.1 wt. % to about 1.0 wt. %. In certain embodiments, the boron content ranges from about 0.5 wt. % to about 1.0 wt. %.

The aluminum content of the boroaluminosilicate molecular sieves prepared as above can range from about 0.01 wt. % to about 3.3 wt. %. In certain embodiments, the aluminum content ranges from about 0.20 wt. % to about 3.3 wt. %; or about 0.3 wt. % to about 2.0 wt. % or about 0.20 wt. % to about 1.5 wt. %. In other embodiments, the boron content ranges from about 0.5 wt. % to about 1.0 wt. % and the aluminum content ranges from a about 0.01 wt. % to about 3.3 wt. %. In yet other embodiments, the boron content ranges from about 0.5 wt. % to about 1.0 wt. % and the aluminum content ranges from about 0.20 wt. % to about 1.5 wt. %. The aluminum content in the MFI framework imparts intrinsic activity to the sieve and therefore eliminates the need for an activation of the borosilicate of the support.

The boroaluminosilicate molecular sieves prepared according to the preceding methods can have average crystallite sizes less than 2 μm, such as, between about 10 nm and about 2 μm. For example, the boroaluminosilicate molecular sieves can have average crystallite sizes ranging from about 50 nm to 1 μm. In certain embodiments, the sieves can have average crystallite sizes ranging from about 100 nm to about 1 μm; or about 50 nm to about 500 nm. In certain embodiments, the sieves can have average crystallite sizes less than about 1 μm. The relatively small size of the sieves is advantageous in that xylene isomerization is diffusion limited with paraxylene having a higher diffusion rate that the other xylene isomers.

The isomerization catalysts used in the methods of the invention can comprise boroaluminosilicate molecular sieves in pure form or may further include a support. Suitable supports include, for example, alumina, (such as Sasol Dispersal® P3 alumina, PHF alumina), titania, and silica, and mixtures thereof. In one embodiment, the support comprises alumina. In another embodiment, the support comprises titania. In another embodiment, the support comprises silica. In another embodiment, the support comprises Sasol Dispersal® P3 alumina.

The support may be provided in a quantity to yield an isomerization catalyst including 1-99 wt. % boroaluminosilicate molecular sieve, such as 10-50 wt. % boroaluminosilicate molecular sieve and the remainder support. In other embodiments, the isomerization catalyst includes 10-30 wt. % boroaluminosilicate molecular sieve and the remainder support. In other embodiments, the isomerization catalyst comprises less than 90 wt. % support; or less than 80 wt. % support; or less than 70 wt. % support; or less than 60 wt. % support; or less than 50 wt. % support; or less than 40 wt. % support; or less than 30 wt. % support; or less than 20 wt. % support; or less than 10 wt. % support; or less than 5 wt. % support.

A hydrogenation catalyst component may be added to the boroaluminosilicate molecular sieve catalysts. Suitable hydrogenation catalyst components include a metal or metal compound with the metals chosen from Groups VI-X of the periodic table. Suitable metals or compounds include, for example, metals or compounds of Pt, Pd, Ni, Mo, Ru, Rh, Re and combinations thereof. In certain embodiments, the hydrogenation catalyst is Mo or a Mo compound. Other promoters or modifiers may be added such as Sn or S. For example, if Pt is used, it may be desirable to alloy with Sn, or to provide a low level of sulfiding.

Again, referring to FIG. 1a, the pX enriched stream (102) produced from the reaction zone (100) may be further processed in a separation zone (120′). The separation zone can include at least a pX recovery zone to recover at least a portion of a pX product (104), and, in certain embodiments, a fractionization zone to recover at least a portion of byproducts, each from the pX enriched stream. Typical byproducts include, for example, transmethylation by products benzene, toluene, trimethylbenzene, methyl(ethyl)benzene, and the like, which may be isolated from the pX enriched stream by standard methods such as fractional distillation. In certain embodiments, the pX enriched stream is processed to recover benzene byproduct and/or toluene byproduct.

Methods for isolating the pX product in the pX recovery zone (120) include, for example, (a) fractional crystallization, (b) liquid phase adsorption to chromatographically separate pX from the other C₈ aromatics; (c) chromatographic separation over zeolite ZSM-5 or ZSM-8, which has been reacted with an organic radical-substituted silane; (d) adsorptive separation of p-xylene and ethylbenzene through the use of ZSM-5 or ZSM-8 zeolites which have been reacted with certain silanes; (e) by heating a mixture of C₈ aromatic hydrocarbons to 50° F.-500° F. (10° C.-260° C.) followed by an adsorption/desorption step in the presence of a molecular sieve or synthetic crystalline aluminosilicate zeolite as the adsorbent (e.g., ZSM-5) to recover a first mixture of p-xylene and ethylbenzene and a second mixture including meta-xylene, ortho-xylene, and any C₉ and higher aromatics; the resulting p-xylene and ethylbenzene mixture can be subjected to crystallization to recover p-xylene and the mother liquor can be subjected to distillation to recover the ethylbenzene; and (0 as disclosed in U.S. Pat. No. 6,573,418, by pressure swing adsorption employing a para-selective adsorbent (e.g., a large crystal, non-acidic medium pore molecular sieve) in connection with simulated moving bed adsorption chromatography.

The pX-lean stream (107) produced from the separation zone (120′) after generation of a pX product (e.g., a reject stream from a crystallization process or a raffinate from an adsorption process), containing relatively high proportions of EB, oX and mX, may be recycled to the reaction zone (100) for use as a hydrocarbon-containing feed stream (101′), or for combination with a hydrocarbon-containing feed stream (101).

As a result of the particular isomerization catalysts, the methods of the invention can provide a pX enriched stream (102) that contains reduced concentrations of byproducts of transmethylation as compared to similar methods using industry-standard xylene isomerization catalysts, such as AMSAC-3200 (20% HAMS-1B-3 borosilicate molecular sieve (hydrogen form of AMS-1B) with 80% alumina binder). For example, the pX enriched stream can contain 3.5 wt. % or less net C₉-byproducts and/or 1.5 wt. % or less net toluene byproduct. The phrase “net byproduct,” refers to weight % of the referenced byproduct in an outgoing stream (e.g., “the pX enriched stream”) less the weight percent of the same “byproduct” in the incoming feed stream (e.g., “hydrocarbon-containing feed stream”). For example, where an incoming hydrocarbon-containing feed stream contains 1 wt. % of a byproduct (e.g., toluene) and the corresponding pX enriched stream contains 5 wt. % of the same byproduct, the pX enriched stream contains 4 wt. % net byproduct (e.g., 4 wt. % net toluene). The term “Cₙ-byproducts” refers to all chemical compounds in the referenced stream or product having “n” carbons in their individual chemical structures. For example, trimethylbenzene is a C₉-byproduct as it contains nine carbons in its chemical structure. In certain embodiments, the byproducts are aromatic compounds. Thus, in certain embodiments, the pX enriched stream can contain 3.5 wt. % or less net C₉-byproducts; or 3.0 wt. % or less; or 2.5 wt. % or less; or 2.0 wt. % or less net C₉-byproducts (e.g., C₉-aromatic byproducts). In other embodiments, the pX enriched stream can contain 1.5 wt. % or less net toluene byproduct; or 1.4 wt. % or less net toluene byproduct; or 1.3 wt. % or less net toluene byproduct; or 1.2 wt. % or less net toluene byproduct; or 1.1 wt. % or less net toluene byproduct; or 1.0 wt. % or less net toluene byproduct; or 0.9 wt. % or less net toluene byproduct; or 0.8 wt. % or less net toluene byproduct.

In other embodiments, the pX enriched stream contains less than 0.7 wt. % net trimethylbenzene byproduct; or less than 0.6 wt. % net trimethylbenzene byproduct or; less than 0.5 wt. % net trimethylbenzene byproduct.

In one embodiment, the present methods provide a pX enriched stream containing at least 23.5 wt. % pX/X. In one embodiment, the pX enriched stream contains at least 23.5 wt. % pX/X and less than 1.5 wt. % net toluene byproduct. In another embodiment, the pX enriched stream contains at least 23.5 wt. % pX/X and less than 1.0 wt. % net toluene byproduct. In another embodiment, the pX enriched stream contains at least 23.8 wt. % pX/X and less than 1.5 wt. % net toluene byproduct. In another embodiment, the pX enriched stream contains at least 23.8 wt. % pX/X and less than 1.0 wt. % net toluene byproduct.

In yet other embodiments, the present methods provide a pX enriched stream containing at least 23.8 wt. % pX/X and less than 0.6 wt. % net trimethylbenzene byproduct. In yet other embodiments, the present methods provide a pX enriched stream containing at least 23.8 wt. % pX/X and less than 0.5 wt. % net trimethylbenzene byproduct.

In further embodiments, the present methods provide a pX enriched stream containing at least 23.5 wt. % pX/X and a ratio of pX/X to the sum of net wt. % trimethylbenzene byproduct and net wt. % toluene byproduct of greater than 4.0 (e.g., between 4.0 and 10.0). In other embodiments, the pX enriched stream contains at least 23.6 wt. % pX/X; or at least 23.7 wt. % pX/X; or at least 23.8 wt. % pX/X and a ratio of pX/X to the sum of net wt. % trimethylbenzene byproduct and net wt. % toluene byproduct of greater than 4.0 (e.g., between 4.0 and 10.0, or between 4.0 and 8.0).

In other embodiments, the pX enriched stream contains at least 23.5 wt. % pX/X; or at least 23.6 wt. % pX/X; or at least 23.7 wt. % pX/X; or at least 23.8 wt. % pX/X and a ratio of pX/X to the sum of net wt. % trimethylbenzene byproduct and net wt. % toluene byproduct of greater than 5.0 (e.g., between 5.0 and 10.0, or between 5.0 and 8.0).

In other embodiments, the pX enriched stream contains at least 23.5 wt. % pX/X; or at least 23.6 wt. % pX/X; or at least 23.7 wt. % pX/X; or at least 23.8 wt. % pX/X and a ratio of pX/X to the sum of net wt. % trimethylbenzene byproduct and net wt. % toluene byproduct of greater than 6.0 (e.g., between 6.0 and 10.0, or between 6.0 and 8.0).

In other embodiments, the pX enriched stream contains at least 23.5 wt. % pX/X; at least 23.6 wt. % pX/X; at least 23.7 wt. % pX/X; or at least 23.8 wt. % pX/X; or essentially equilibrium pX concentration for the temperature of the reaction (e.g., 24.1 wt. % at between 700° F. and 750° F.).

In certain embodiments, as shown in FIG. 1b, the pX enriched stream (102) produced from the reaction zone can be further processed in a fractionization zone (110) to recover at least a portion of the byproducts (103) from the pX enriched stream. Typical byproducts and methods for isolation can be as described above. In certain embodiments, the pX enriched stream (102) is processed in the fractionization zone (110) to recover benzene byproduct and/or toluene byproduct. After removal of byproducts, at least a portion of the pX product (104) can be recovered in a pX recovery zone (120) from the pX enriched stream (102). The pX-lean stream (107) produced after generation of a pX product may be recycled to the reaction zone (100) for use as a hydrocarbon-containing feed stream (101′), or for combination with a hydrocarbon-containing feed stream (101).

Referring to FIG. 1c, in another embodiment, prior to recovery of the pX product (104), the pX enriched stream (102) may be combined with a make-up feed stream (105). The make-up feed stream (105) may be introduced, as shown by branch (105a), at the fractionation zone (110) to provide a combination stream (106) from the fractionation zone. The make-up feed stream (105a) provided to the fractionation zone (110) can be, for example, a C8+ reformate distillation cut of a refinery reformer. In this case, the fractionation zone (110) can remove byproducts (103) produced in reaction zone (100) and C9+ aromatics or other non-C8 aromatics that may be present in make-up feed stream (105). Alternatively, depending on the source of the make-up feed stream (e.g., where byproduct removal is not necessary), the make-up feed stream (105) may be introduced, as shown by branch (105b), after the fractionation zone (110) to provide the combination stream (106). Then, at least a portion of the pX product (104) may be recovered from the combination stream (106) in a recovery zone (120). The resulting pX-lean stream (107) can be recycled in any of the preceding methods to the reaction zone (100) for use as the hydrocarbon-containing feed stream (101′), or for combination with a hydrocarbon-containing feed stream (101).

Thus, in one embodiment, as shown in FIG. 1c, a reaction zone (100) comprises a reactor with a catalyst or dual bed catalyst system comprising a boroaluminosilicate molecular sieve prepared according to this invention. The reaction zone (100) isomerizes the xylenes and converts some of the ethylbenzene in the hydrocarbon-containing feed stream (101 or 101′) producing a pX enriched stream (102), while producing some byproducts including benzene, toluene and A9+ aromatics. At least a portion of the byproducts produced are separated in fractionation zone (110) to produce byproducts stream(s) (103). The pX enriched stream freed of some byproducts is combined with a make-up feed stream (105b) comprising the xylene isomers and ethylbenzene to produce a combination stream (106) which is fed to a pX recovery zone (120). Alternatively, a make-up stream (105a), for example, a C8+ reformate distillation cut of a refinery reformer, is fed to the fractionation zone (110), and the combination stream (106) produced from the fractionation zone. Then, at least a portion of the pX in the combination stream (106) is removed in a pX recovery zone (120) as a pX product stream (104). The pX recovery zone (120) also produces a pX lean stream (107) which is recycled to reaction zone (100) as the hydrocarbon-containing stream (101) or for combination with a hydrocarbon-containing stream (101′).

The preceding methods may be practiced in conjunction with a dual-bed catalyst configuration. Accordingly, the methods may further include contacting the hydrocarbon-containing feed stream with an ethylbenzene (EB) conversion catalyst under conditions suitable to reduce the EB content of the hydrocarbon-containing feed stream. Such contacting may occur, for example, prior to contacting the hydrocarbon-containing feed stream with the isomerization catalyst. In certain embodiments, the hydrocarbon-containing feed stream is contacted with the EB conversion catalyst and the isomerization catalyst in a single reaction zone.

Suitable ethylbenzene conversion catalysts include, for example, AI-MFI molecular sieve dispersed on silica and large particle size molecular sieves, such as ZSM-5 molecular sieve having a particle size of at least about 1 μm, dispersed on silica, alumina, silica/alumina or other suitable support. In one example, the EB conversion catalyst includes an Al-MFI molecular sieve having a particle size of at least about 1 μm supported on Cab-o-Sil® HS-5 (a high surface fumed silica available from Cabot Corporation, Billerica, Mass.) with a compound of Mo added. Suitable catalysts based on a ZSM-type molecular sieve, for example, ZSM-5 molecular sieves. In addition, other types of molecular sieve catalysts can also be used (e.g., ZSM-11, ZSM-12, ZSM-35, ZSM-38 and other similar materials).

As noted, a hydrogenation catalyst component may be added to the ethylbenzene conversion catalyst, with the hydrogenation catalyst being a metal or metal compound with the metals chosen from Groups VI-X of the periodic table, as noted above for the isomerization catalysts. In certain embodiments, the hydrogenation catalyst is Mo or a Mo compound. Other promoters or modifiers may be added such as Sn or S. For example, if Pt is used, it may be desirable to alloy with Sn, or to provide a low level of sulfiding. In other embodiments, both the isomerization catalyst and the ethylbenzene conversion catalyst comprise a hydrogenation catalyst. In certain embodiments, both catalysts comprise Mo or a Mo compound.

The ethylbenzene conversion catalyst may include about 1% to about 100% by weight of molecular sieve, or about 10 to about 70% by weight, with the remainder being support matrix material such as alumina or silica, or a mixture thereof. In certain embodiments, the support material is silica. In certain embodiments, the support material is alumina. In certain embodiments the support is a combination of silica and alumina. The weight ratio of ethylbenzene conversion catalyst to isomerization catalyst can be about 0.25:1 to about 6:1.

Catalyst Systems

In another aspect, the present invention provides catalyst system for use in any of the preceding methods and embodiments of the same. In particular, the catalyst systems are useful in methods for enriching a xylene isomers feed in p-xylene. Such catalyst systems include dual bed configurations including a first bed including an ethylbenzene (EB) conversion catalyst and a second bed including an isomerization catalyst including a boroaluminosilicate molecular sieve.

The boroaluminosilicate molecular sieves can be prepared according to methods familiar to those skilled in the art. For example, boroaluminosilicate molecular sieves can be prepared by, first, combining a boron source, an aluminum source, a silica sol, a template, and a base to form a reaction mixture.

The boron source may be any familiar to one skilled in the art for preparing molecular sieves, including for example boric acid. The silica sol can be commercially available colloidal silicas, for example, Ludox® HS-40 (40 wt. % suspension of colloidal silica in H₂O), Ludox® AS-40 (40 wt. % suspension of colloidal silica in H₂O, stabilized by ammonium hydroxide), and Nalco 2327, among others. NALCO 2327 has a mean particle size of 20 nm and a silica content of approximately 40 percent by weight in water with a pH of approximately 9.3, and ammonium as the stabilizing ion. Methods of making colloidal silica particles include, for example, partial neutralization of an alkali-silicate solution.

The aluminum source can be sodium aluminate, or can be alkali free, such as aluminum sulfate, aluminum nitrate, an aluminum C₁₋₁₀alkanoate, or an aluminum C₁₋₁₀alkoxide such as aluminum isopropoxide. The template may be any familiar to one skilled in the art for preparing molecular sieves, including for example tetra(C₁₋₁₀alkyl)ammonium compounds, such as tetra(C₁₋₁₀alkyl)ammonium hydroxide (e.g., tetra(propyl)ammonium hydroxide) or a tetra(C₁₋₁₀alkyl)ammonium halide (e.g., tetra(propyl)ammonium bromide).

The base can be either a Brønsted or Lewis base that, when dissolved in water, yields a basic solution (i.e., pH>7). That is, the present invention excludes boroaluminosilicate molecular sieves prepared using ammonium fluoride to facilitate the formation of the molecular sieves. In certain embodiments, the base is an alkali metal base or an alkaline earth metal base, such as, for example NaOH, KOH, Ca(OH)₂, and the like. In certain other embodiments, the base is an essentially metal-free base, such as, for example, ammonium hydroxide.

The reaction mixture is warmed to provide a product mixture containing a solid. Suitably, the reaction mixture can be warmed to a temperature between 100° C. and 200° C.; or to a temperature between 150° C. and 170° C., for a suitable time to provide the product mixture containing the solid. For example, the reaction mixture can be heated to a suitable temperature in an autoclave at autogenous pressure. The solid is isolated from the product mixture, for example, by filtration or centrifugation.

Where the boroaluminosilicate molecular sieve is prepared using a base that contains alkali metal cations (e.g., Na⁺) and/or alkali earth cations (e.g., Mg²⁺), and/or using an alkali metal containing aluminum source (e.g., sodium aluminate), and/or using a silica sol stabilized by an alkali metal source, the solid can be contacted with a cation exchange solution containing an ammonium salt, such as ammonium acetate, in an amount and for a period of time suitable to exchange the alkali metal cations and/or alkali earth cations for hydrogen (i.e., to provide the H⁺-form of the boroaluminosilicate molecular sieve). However, the use of an amine base, as defined above, for the preparation of the boroaluminosilicate molecular sieve can avoid the necessity of cation exchange.

Ultimately, the resulting solid, with or without cation exchange, can be calcined to yield the boroaluminosilicate molecular sieve. The calcining is typically at a temperature between 480° C. and 600° C.

The boroaluminosilicate molecular sieves prepared according to the preceding methods typically have an MFI framework and can have an alkali metal content less than 400 ppmw (e.g., between about 10 ppmw and about 400 ppmw). In certain embodiments, the boroaluminosilicate molecular sieve has an alkali metal content is less than 350 ppmw (e.g., between about 10 ppmw and about 350 ppmw); or less than 300 ppmw (e.g., between about 10 ppmw and about 300 ppmw); or less than 250 ppmw (e.g., between about 10 ppmw and about 250 ppmw); or less than 200 ppmw (e.g., between about 10 ppmw and about 200 ppmw); or less than 150 ppmw (e.g., between about 10 ppmw and about 150 ppmw). In certain other embodiments, the boroaluminosilicate molecular sieve has an alkali metal content of less than 100 ppmw (e.g., between about 10 ppmw and about 110 ppmw).

The boron content of the boroaluminosilicate molecular sieves prepared as above can range from about 0.01 wt. % to about 1.5 wt. %. In certain embodiments, the boron content ranges from about 0.01 wt. % to about 1.2 wt. %; or about 0.01 wt. % to about 1.0 wt. %; or about 0.1 wt. % to about 1.0 wt. %. In certain embodiments, the boron content ranges from about 0.5 wt. % to about 1.0 wt. %.

The aluminum content of the boroaluminosilicate molecular sieves prepared as above can range from about 0.01 wt. % to about 3.3 wt. %. In certain embodiments, the aluminum content ranges from about 0.20 wt. % to about 3.3 wt. %; or about 0.3 wt. % to about 2.0 wt. % or about 0.20 wt. % to about 1.5 wt. %. In other embodiments, the boron content ranges from about 0.5 wt. % to about 1.0 wt. % and the aluminum content ranges from a about 0.01 wt. % to about 3.3 wt. %. In yet other embodiments, the boron content ranges from about 0.5 wt. % to about 1.0 wt. % and the aluminum content ranges from about 0.20 wt. % to about 1.5 wt. %.

The boroaluminosilicate molecular sieves prepared according to the preceding methods can have average crystallite sizes less than 2 μm, such as, between about 10 nm and about 2 μm. For example, the boroaluminosilicate molecular sieves can have average crystallite sizes ranging from about 50 nm to 1 μm. In certain embodiments, the sieves can have average crystallite sizes ranging from about 100 nm to about 1 μm; or about 50 nm to about 500 nm. In certain embodiments, the average crystallite size is less than about 1 μm.

The isomerization catalysts used in the methods of the invention can comprise boroaluminosilicate molecular sieves in pure form or may further include a support. Suitable supports include, for example, alumina (such as Sasol Dispersal® P3 alumina or PHF alumina), titania, and silica, and mixtures thereof. In one embodiment, the support comprises alumina. In another embodiment, the support comprises titania. In another embodiment, the support comprises silica. In another embodiment, the support comprises Sasol Dispersal® P3 alumina.

The support may be provided in a quantity to yield an isomerization catalyst including 1-99 wt. % boroaluminosilicate molecular sieve, such as 10-50 wt. % boroaluminosilicate molecular sieve and the remainder support. In other embodiments, the isomerization catalyst includes 10-30 wt. % boroaluminosilicate molecular sieve and the remainder support. In other embodiments, the isomerization catalyst comprises less than 90 wt. % alumina; or less than 80 wt. % alumina; or less than 70 wt. % alumina; or less than 60 wt. % alumina; or less than 50 wt. % alumina; or less than 40 wt. % alumina; or less than 30 wt. % alumina; or less than 20 wt. % alumina; or less than 10 wt. % alumina; or less than 5 wt. % alumina.

A hydrogenation catalyst component may be added to the boroaluminosilicate molecular sieves, with the hydrogenation catalyst being a metal or metal compound with the metals chosen from Groups VI-X of the periodic table. Suitable metals or compounds include, for example, metals or compounds of Pt, Pd, Ni, Mo, Ru, Rh, Re and combinations thereof. In certain embodiments, the hydrogenation catalyst is Mo or a Mo compound. Other promoters or modifiers may be added such as Sn or S. For example, if Pt is used, it may be desirable to alloy with Sn, or to provide a low level of sulfiding.

Suitable ethylbenzene conversion catalysts include, for example, AI-MFI molecular sieve dispersed on silica and large particle size molecular sieves, such as ZSM-5 molecular sieve having a particle size of at least about 1 μm, dispersed on silica, alumina, silica/alumina or other suitable support. In one example, the EB conversion catalyst includes an Al-MFI molecular sieve having a particle size of at least about 1 μm supported on Cab-o-Sil® HS-5 (a high surface fumed silica available from Cabot Corporation, Billerica, Mass.) with a compound of Mo added. Suitable catalysts based on a ZSM-type molecular sieve, for example, ZSM-5 molecular sieves. In addition, other types of molecular sieve catalysts can also be used (e.g., ZSM-11, ZSM-12, ZSM-35, ZSM-38 and other similar materials).

As noted, a hydrogenation catalyst may be added to the ethylbenzene conversion catalyst, with the hydrogenation catalyst being a metal or metal compound with the metals chosen from Groups VI-X of the periodic table, as noted above for the isomerization catalysts. In certain embodiments, the hydrogenation catalyst is Mo or a Mo compound. Other promoters or modifiers may be added such as Sn or S. For example, if Pt is used, it may be desirable to alloy with Sn, or to provide a low level of sulfiding. In other embodiments, both the isomerization catalyst and the ethylbenzene conversion catalyst comprise a hydrogenation catalyst. In certain embodiments, both catalysts comprise Mo or a Mo compound.

The ethylbenzene conversion catalyst may include about 1% to about 100% by weight of molecular sieve, or about 10 to about 70% by weight, with the remainder being support matrix material such as alumina or silica, or a mixture thereof. In certain embodiments, the support material is silica. In certain embodiments, the support material is alumina. The weight ratio of ethylbenzene conversion catalyst to isomerization catalyst is suitably about 0.25:1 to about 6:1.

In certain embodiments, the first bed, including the EB conversion catalyst is disposed over the second bed, including the boroaluminosilicate molecular sieve. The phrase “disposed over” means that the first referenced item (e.g., first bed) can be in direct contact with the surface of the second referenced item (e.g., second bed), or one or more intervening materials or structures may also be present between the surface of the first item (e.g., first bed) and the surface of the second item (e.g., second bed). However, when one or more intervening materials or structures are present (such as screens to support and/or separate the first and second beds), the first and second items, nonetheless, remain in fluid communication with each other (e.g., the screens allow for the hydrocarbon-containing feed stream to pass from the first bed to the second bed). Further, the first item (e.g., first bed) may cover the entire surface or a portion of the surface of the second item (e.g., second bed). Alternatively, the catalyst system includes a guard bed, including a hydrogenation catalyst, disposed over the first bed. A guard bed may also be disposed between the first bed and the second bed. The weight ratio of ethylbenzene catalyst to hydrogenation catalyst can be about 1:1 to about 20:1.

The hydrogenation catalyst may contain a hydrogenation metal, such as molybdenum, platinum, palladium, rhodium, ruthenium, nickel, iron, osmium, iridium, tungsten, rhenium, and the like, and may be dispersed on a suitable matrix. Suitable matrix materials include, for example, alumina and silica. Although a molybdenum-on-alumina catalyst is effective, other hydrogenation catalysts, for example those including platinum, palladium, rhodium, ruthenium, nickel, iron, osmium, iridium, tungsten, rhenium, etc., deposited on a suitable support such as alumina or silica may also be used. It is advantageous to avoid hydrogenation catalysts and/or reaction conditions that cause aromatic ring hydrogenation of the xylenes. When molybdenum-on-alumina is used, the level of molybdenum can be about 0.5 to about 10 weight percent, or about 1 to about 5 weight percent.

In another aspect, the invention provides xylene isomerization reactor including a reaction zone containing the catalyst system as described above. The xylene isomerization reactor can be a fixed bed flow, fluid bed, or membrane reactor containing the catalyst system described above. The reactor can be configured to allow a hydrocarbon-containing feed stream to be cascaded over the catalyst system disposed in a reaction zone in sequential beds; for example, first, the EB conversion catalyst bed and then the xylene isomerization catalyst bed; or first, the xylene isomerization catalyst and then the EB conversion catalyst. In another embodiment, first, the EB conversion catalyst bed, then, a “sandwiched” hydrogenation catalyst bed, and finally, the xylene isomerization catalyst bed. Alternatively, first, the xylene isomerization catalyst bed, then, the “sandwiched” hydrogenation catalyst bed, and finally, the EB conversion catalyst bed. In another embodiment, the reactor may include separate sequential reactors wherein the feed stream would first be contacted with the EB conversion catalyst in a first reactor, the effluent from there would be optionally contacted with the “sandwiched” hydrogenation catalyst in an optional second reactor, and the resulting effluent stream would then be contacted with the xylene isomerization catalyst in a third reactor. In another embodiment, the xylene isomerization catalyst bed may comprise a hydrogenation catalyst disposed over the EB conversion catalyst and another “sandwiched” hydrogenation catalyst between the EB conversion catalyst and the isomerization catalyst.

While specific embodiments have been described in detail, and in particular in the following Examples, those with ordinary skill in the art will appreciate that various modifications and alternatives could be developed in light of the overall teachings of the disclosure. Accordingly, the particular arrangements disclosed are meant to be illustrative only and not limiting as to the scope of the invention, which is to be given the full breadth of the appended claims, including any and all equivalents thereof. Unless otherwise defined, all technical and scientific terms used herein have the same meaning as commonly understood by one of ordinary skill in the art to which this invention pertains. All references mentioned in this description, including publications, patent applications, and patents, are incorporated by reference in their entirety. In addition, the materials, methods, and examples described are only illustrative and not intended to be limiting.

### EXAMPLES

### Example 1

### Preparation of Conventional and Boroaluminosilicate Molecular Sieves

### (a) General Preparation

Precursors such as silica sol, an aluminum compound, tetrapropylammonium template, and base were mixed and charged into 125-cc Parr reactors. These reactors were sealed and then heated at 150-170° C. for 2-5 days in an oven. Agitation of the reactor contents was accomplished by rotational tumbling of the reactors inside the temperature-controlled oven. The oven could accommodate up to 12 reactors simultaneously. Product work-ups involved standard filtration, water-washing, and drying methods. Final products were typically calcined at 538° C. (1000° F.) for 5 hours.

### (b) “Conventional” ZSM-5 Aluminosilicates

“Conventional” ZSM-5 aluminosilicates were made using an aqueous mixture of the silica sol, aluminum sulfate or sodium aluminate, template (tetrapropylammonium bromide), and base (NaOH), followed by ammonium acetate exchange to remove sodium.

### (c) Boroaluminosilicates

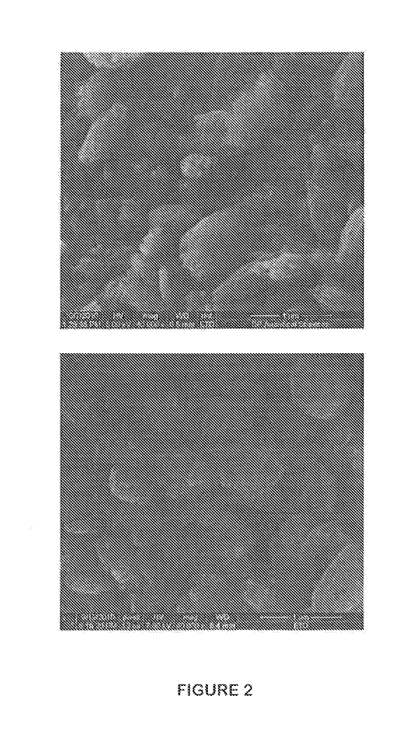

Boroaluminosilicates were prepared using an aqueous mixture of silica sol, aluminum sulfate, boric acid, template (tetrapropylammonium bromide), base (ethylenediamine), and heated at 150-170° C. for 3-5 days. Since these boroaluminosilicate sieves were prepared using ethylenediamine as the base instead of sodium hydroxide, and thus were low in sodium content, no ammonium acetate exchange was needed. Product work-ups involved standard filtration, water-washing, and drying methods. Example SEM images of a boroaluminosilicates prepared using ethylenediamine as a base are shown in FIG. 2. The sieves of FIG. 2 have average particle sizes in the long direction of less than about 1 micron.

### Example 2

### Comparative Catalytic Activity Study

Samples of “commercial” zeolite molecular sieves and catalysts were obtained from Tosoh, Zeolyst, TriCat, Qingdao Wish Chemical, and Zibo Xinhong Chemical Trade Co (see Table 1). The TriCat and Tosoh “HSZ-820NAA” samples were ammonium-exchanged by a conventional procedure: an ammonium acetate solution was made by dissolving 1 g ammonium acetate in 10 g deionized (DI) water (such as 100 g ammonium acetate in 1000 g DI water). Then 1 g of the sieve to be exchanged was added to 11 g of the ammonium acetate solution. The mixture was heated to 85° C. for one hour while stirring, filtered using a vacuum filter, and washed with 3 aliquots of 3 g DI water per g of sieve while the sieve was still on the filter paper. The sieve was re-slurried in 11 g of fresh ammonium acetate solution, heated to 85° C. on a heating pad for one hour while stirring, filtered and washed with DI water as per above. It was then dried and calcined in air: 4 hrs at 329° F., ramp to 900° F. over 4 hours, calcined for 4 h. at 900° F.

Commercial ZSM-5 aluminosilicate catalysts and boroaluminosilicate molecular sieves were tested unsupported (i.e., as “pure” sieves) and were supported on alumina (20% sieve, 80% alumina) according to the following procedure:

40 g Sasol Disperal® P3 alumina (Sasol Germany GmbH, Hamburg, Germany) was added to 360 g of 0.6 wt % deionized distilled (DD) water to form an alumina sol, and homogenized for 15 minutes. A mixture of 8 g of sieve in 24 g DD water was prepared and homogenized for 3 minutes. 320 g of the alumina sol was placed into a beaker and the sieve/DD water mixture was added, followed by homogenization for 5 minutes. After standing for 30 minutes, the sieve/sol mixture was transferred to a kitchen blender and 24 mL of concentrated ammonium hydroxide (nominal 28 wt % ammonia) was added. The resulting gel was mixed at setting 4 for 5 minutes. The mixture was poured into a drying dish (about 2 inch depth), dried for 4 h. at 329° F., ramped to 900° F. over 4 hours, and finally calcined at 900° F. for 4 hours.

The following catalysts were prepared as controls:
1. “AMSAC-3200P3” containing nominal 20 wt. % HAMS-1B-3 borosilicate molecular sieve (hydrogen form of AMS-1B) and 80 wt. % Sasol Disperal® P3 alumina
2. “AMSAC-3200”, commercial, nominal 20 wt. % borosilicate molecular sieve with 80 wt. % alumina binder.
3. “AMSAC-3202M”, commercial, nominal 20 wt. % borosilicate molecular sieve with 80 wt. % alumina binder, contains 2 wt. % Mo.

Catalytic Testing

The catalysts were charged into 2-mm ID tube reactors as powders (50 μm-200 μm) in a high-throughput catalyst testing apparatus consisting of 16 parallel fixed-bed flow reactors. The catalysts were activated by heating the reactors under H₂ flow without hydrocarbon feed for at least an hour at reaction temperature prior to introducing hydrocarbon feed. Then, hydrogen gas and the xylene isomers were combined and fed to the reactor. Reactor effluent hydrocarbons were analyzed every 4 hours by an on-line gas chromatograph.

The feed stream of xylene isomers contained 1.03 wt. % benzene, 1.98 wt. % toluene, 10.57 wt. % EB (ethylbenzene), 9.75 wt. % pX (p-xylene), 50.22 wt. % mX (m-xylene), and 24.16 wt. % oX (o-xylene), corresponding to 11.6% pX isomer in the xylene isomers.

A first testing phase was conducted to screen and rank catalysts for xylene isomerization activity. Relatively mild conditions were employed (600° F., 38 h⁻¹ WHSV xylenes feed, 225 psig, 1.5 H₂/hydrocarbon mole ratio and LWHSV=38 based on 20 wt % sieve catalysts with LWHSV adjusted based on sieve content when testing unsupported sieves) to discriminate based on activity for xylene isomerization. EB conversions were very low, <10%, under these mild conditions. Isomerization of xylenes to theoretical equilibrium would yield about 24.1% pX/xylenes in the reactor effluent. Reactor effluents were sampled periodically during the runs and analyzed by gas chromatography. Catalysts were observed to undergo moderate deactivation over 50+ hours on stream. Due to the deactivation, % pX/xylenes results were calculated as averages over the first 40-50 hours on stream.

Each run (block of 16 reactors) included at least two of the AMSAC-3200 and/or AMSAC-3202M reference catalysts as controls. The performance of the AMSAC references was reproducible from run to run

Of the 60 catalysts tested, 17 were found to isomerize xylenes with similar effectiveness as the AMSACs (20-23% pX/xylenes), including 12 commercial ZSM-5 materials and the boroaluminosilicates. Five other catalysts (ZSM-5 and alumina-supported boroaluminosilicates) exhibited slightly lower isomerization activity (19% pX/xylenes) than the AMSACs. The remaining catalysts were less active, with about a dozen being essentially inactive. Table 1 presents a summary of the most active catalysts in the first phase of testing, where “S” indicates the sieve was tested in pure form and “C” indicates that the sieve was supported on alumina, as prepared above.

| | | % pX/ | Sieve (S) or | | |
|---|---|---|---|---|---|
| | | Xylenes | Alumina- | Al | |
| | | (avg of 2 | supported | wt % | B |
| Catalyst | Type | trials) | (C) | in sieve | wt % |
| Control Catalysts | | | | | |
| AMSAC-3200 | borosilicate | 21% | C | | |
| AMSAC-3200 P3 | borosilicate | 22% | C | | |
| AMSAC-3202M | borosilicate | 21% | C | | |
| Commercial Catalysts | | | | | |
| Tosoh (Grove City, OH) | | | | | |
| HSZ ®-820NAA, H-form | ZSM-5 | 21% | S | 3.4 | |
| HSZ ®-820NAA, H-form | ZSM-5 | 23% | C | 3.4 | |
| Zeolyst Int'l. | | | | | |
| (Conshohocken, PA) | | | | | |
| CBV 2314 | ZSM-5 | 20% | S | 3.4 | |
| CBV 2314CY1.6 | ZSM-5 | 20% | S | 3.4 | |
| CBV 3024E | ZSM-5 | 21% | S | 2.5 | |
| CBV 3024E | ZSM-5 | 22% | C | 2.5 | |
| CBV 3014CY1.6 | ZSM-5 | 21% | C | 2.5 | |
| CBV 5524G | ZSM-5 | 20% | S | 1.5 | |
| TriCat Catalysts (Hunt | | | | | |
| Valley, MD) | | | | | |
| TriCat | ZSM-5 | 21% | S | 3.3 | |
| TriCat | ZSM-5 | 23% | C | 3.3 | |
| Zibo Xinhong Chemical | | | | | |
| (Zibo City, China) | | | | | |
| Zibo Xinhong Chemical | ZSM-5 | 21% | S | 2.5 | |
| Zibo Xinhong Chemical | ZSM-5 | 22% | C | 2.5 | |
| Example 1(b) | ZSM-5 | 23% | S | 2.2 | |
| Example 1(b) | ZSM-5 | 19% | S | 2.2 | |
| Example 1(b) | ZSM-5 | 19% | S | 2.2 | |
| Example 1(c) | B—Al—SiOx | 20% | C | 0.4 | 0.7 |
| Example 1(c) | B—Al—SiOx | 19% | C | 0.2 | 0.9 |
| Example 1(c) | B—Al—SiOx | 21% | C | 0.7 | 0.7 |
| Example 1(c) | B—Al—SiOx | 23% | C | 1.3 | 0.5 |

Many of the ZSM-5 catalysts were active in their unsupported, pure sieve form. In contrast, the boroaluminosilicates were less active in pure form but were substantially activated by supporting on alumina. This is similar to the behavior of borosilicate catalysts for xylene isomerization. The most active boroaluminosilicate sieve yielded only 16% pX/xylenes in pure sieve form but 23% pX/xylenes in alumina-supported form (20% sieve/80% alumina). This particular boroaluminosilicate had the highest Al content (1.3 wt. %) of all the boroaluminosilicates screened in this study.

### Example 3

### Commercial Conditions Testing

Based on the results of Example 2, approximately thirty isomerization catalysts were tested at higher temperatures (650° F.-770° F.) that are more typical of a commercial PX reactor, to determine isomerization activity and selectivity at higher EB conversions (20-70%). For selectivity, the extent of xylene loss reactions through transmethylation processes was measured, such as the methyl transfer reactions.

Data was collected at five different temperatures (650° F., 680° F., 710° F., 740° F., 770° F.) at 10 h⁻¹ WHSV xylenes feed, 225 psig, and 1.5 H₂/hydrocarbon mole ratio. Typically, three reactor effluent samples were taken at each temperature and analyzed by gas chromatography. Averages of the three sample analyses were calculated.

Ethylbenzene conversions were observed at each of the five tested temperatures. In general, it was observed that the commercial and conventionally-made ZSM-5 sieves showed the highest activity for EB conversion, the AMSAC references and boroaluminosilicates exhibited lower activity. In contrast, activities for xylene isomerization were nearly the opposite. The commercial and conventionally-made ZSM-5 sieves displayed significantly lower isomerization activities than most of the other catalysts. The best catalysts (AMSACs. and most of the boroaluminosilicates) isomerized the xylenes to about 23.9-24.0% pX, near thermodynamic equilibrium (24.1% pX).

Viewed in terms of EB conversion versus xylene isomerization activity, the commercial and conventionally-prepared ZSM-5 aluminosilicate catalysts were largely inferior to the other catalyst groups, including the boroaluminosilicates, in xylene isomerization activity over a wide range of EB conversions.

Catalyst selectivity was examined by comparing the relative amounts of undesirable products generated through transmethylation reactions. Toluene is produced through two transmethylation reactions: xylene disproportionation and methyl transfer from xylene (XYL) to EB. Other transmethylation products include trimethylbenzenes (TMB) and methylethylbenzenes (MEB). For catalysts containing hydrogenation catalysts, toluene (TOL) can also be formed from secondary dealkylation of MEB:

$$XYL + EB = MEB + TOL$$

$$\frac{MEB + {H}_{2} \text{--} > TOL + {C}_{2}}{XYL + EB + {H}_{2} \text{--} > 2 TOL + {C}_{2}} ( Net Reaction )$$

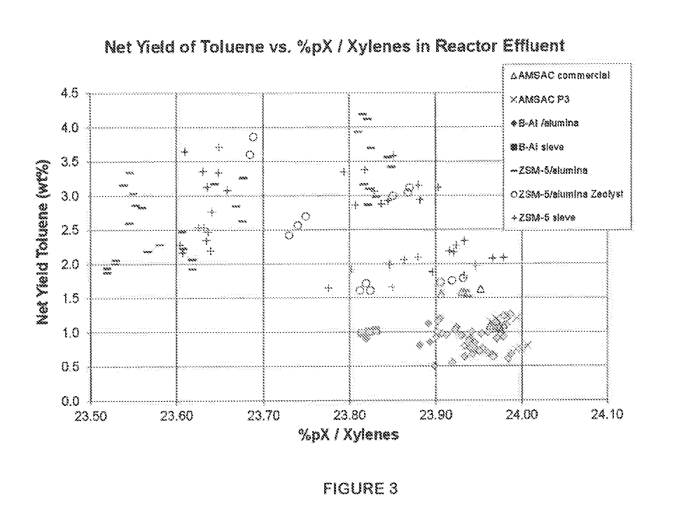

The amount of toluene in the reactor effluent (GC area %) was examined over a range of EB conversions for the catalyst groups. The AMSACs and the boroaluminosilicates yielded very similar and low amounts of toluene, whereas the commercial and conventionally-prepared ZSM-5 aluminosilicate catalysts yielded substantially more toluene. FIG. 3 is a graph of net toluene yield (toluene in feed has been subtracted out) as a function of xylene isomerization activity. Again, the AMSACs and the boroaluminosilicates yielded lower amounts of toluene relative to the other ZSM-5 aluminosilicate catalysts.

Of particular interest is the data shown for the one boroaluminosilicate that was tested as an unsupported sieve (1.3% Al, three squares in FIG. 3), indicating reasonably good isomerization activity and relatively low toluene yield despite not being activated by alumina support.

With respect to other byproducts, trimethylbenzenes and methylethylbenzenes, most of the commercial and conventionally-prepared ZSM-5 aluminosilicate catalysts yielded higher amounts of these than did the AMSACs and the boroaluminosilicate catalysts.

In summary, at the higher temperature conditions, the boroaluminosilicate molecular sieves exhibited high xylene isomerization activity (23.9-24.0% pX/xylenes) that was very similar to the performance of AMSAC-3200 reference catalysts. The boroaluminosilicates also produced low xylene losses from transmethylation reactions (to toluene, trimethylbenzenes, and methylethylbenzenes) over a wide range of EB conversions (20-70%), also similar to the performance of AMSAC-3200 reference catalysts. In contrast, the commercial and conventionally-prepared ZSM-5 catalysts performed poorly and showed relatively low isomerization activity under these conditions (less than 23.9% PX/xylenes) and higher activity for undesirable xylene transalkylation (xylene loss) reactions.

### Example 4

### Quantification of Byproducts

Catalysts were tested for isomerization of xylenes using small fixed-bed flow reactors with a commercial “xylene isomers” aromatics feed consisting of 1.03 wt. % benzene, 1.98% toluene, 10.57% ethylbenzene, 9.75% p-xylene, 50.22% m-xylene, and 24.16% o-xylene (11.6% p-xylene in total xylenes). The catalysts were charged into 2-mm ID tube reactors as powders (50 μm-200 μm). Hydrogen gas and the xylene isomers were combined and fed to the reactor in a 1.5 mole ratio (H₂/hydrocarbon) at 225 psig and with a xylene isomers feed rate of 10 LWHSV (gm feed/gm catalyst-hr). Reactor temperature was either 650° F. or 680° F. Reactor effluent hydrocarbons were analyzed every 4 hours by an on-line gas chromatograph.

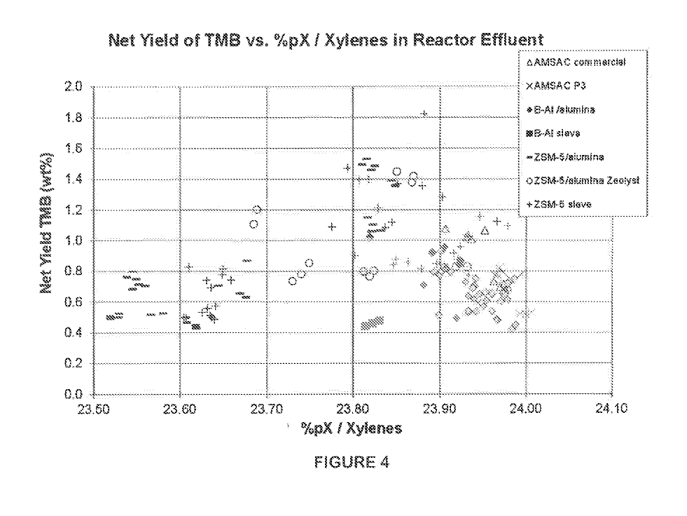

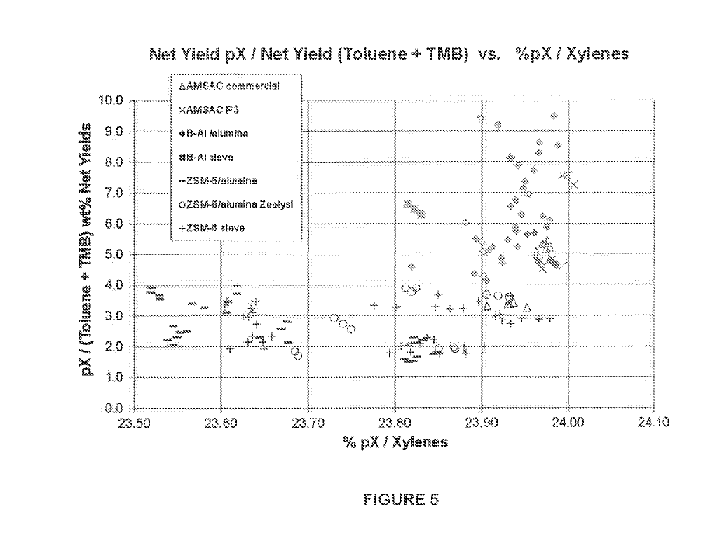

The catalysts were compared over a narrow temperature range (650° F. or 680° F.) and at similar ethylbenzene conversions (32-38%). The results indicate that the boroaluminosilicate molecular sieves produced significantly lower yields of undesired transmethylation products (toluene, trimethylbenzene (TMB), and methylethylbenzene (MEB)) than the commercial catalysts (as shown in FIGS. 4 and 5). If fact, yields of these undesired products were typically about one-half those of the commercial catalysts. In addition, the boroaluminosilicate molecular sieves were highly active for xylene isomerization, yielding at least 23.9% p-xylene isomer in the effluent xylenes.

### Example 5

### Pilot Plant Trials

Catalysts were tested at pilot plant scale under various conditions for the isomerization of xylenes using a “xylene isomers” aromatics feed comprising a total xylene isomers content of from about 83.9 to about 85.6 wt % total xylene and having a pX/X of from about 11.3% to about 11.8%. These pilot plant scale catalyst screening runs typically used 4 gm of catalyst.

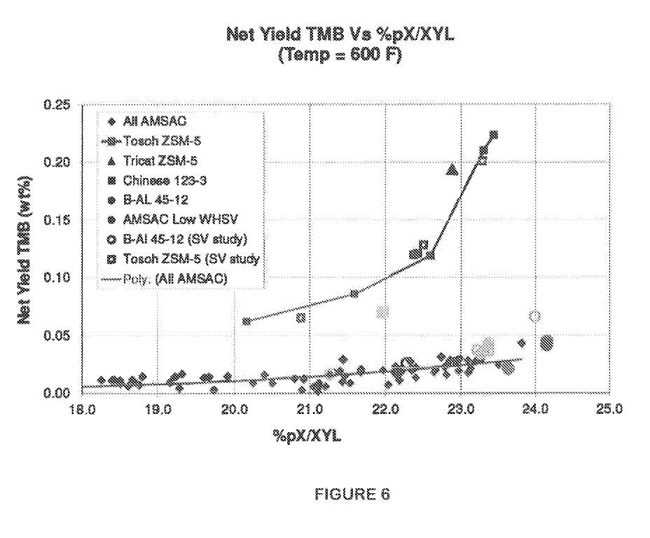

The testing results are shown in FIG. 6. The diamonds are for AMSAC-3200 catalysts comprising of nominal 20 wt % HAMS-1B-3 borosilicate molecular sieve on alumina catalysts prepared via various preparation techniques and using different alumina sources. All of these catalysts were tested at nominal conditions of T=600 F, P=250 psig, H2/Hc=1.5 and LWHSV=38. The open circles are for a catalyst containing a boroaluminosilicate on an alumina support and obtained during a variable study where LWHSV was varied from 8.5 to 80 (gm feed/gm catalyst-hr), with other nominal variables being a reactor temperature of 600° F., reactor pressure of 250 psig, and a 1.5 mole ratio of H₂ to hydrocarbon feed (the solid light gray circle was a run at LWHSV=38). The boroaluminosilicate molecular sieve had an aluminum content of 1.3 wt % and a boron content of 0.48 wt %. The pX/X in the reactor effluent for catalysts comprising a Tosoh ZSM-5 aluminosilicate having an Al content of about 3.4 wt % and a SiO₂/Al₂O₃ ratio of about 23.8 was varied in two ways in FIG. 6. For the solid squares, pX/X was varied by using catalysts with different sieve on alumina content (10 wt. %, 15 wt. %, 20 wt. %, 40 wt. %, and 50 wt. % ZSM-5 on P3 alumina). For the open squares, pX/X was varied by changing contact time (LWHSV=8.5, 10, 20, 38, 60, and 80) at constant nominal conditions of T=600° F., P=250 psig, H2/Hc=1.5. The light gray solid box and the dark gray solid circles were catalysts comprising about 20 wt. % of commercial ZSM-5 sieves from TriCat and a Chinese supplier on P3 alumina.

This example shows that a nominal 20 wt. % boroaluminosilicate (prepared as described in this application) on alumina catalyst provides high xylene isomerization activity with low net trimethylbenzene byproduct production. This makes the xylene isomerization activity of boroaluminosilicate molecular sieve catalysts of this application comparable to that of standard borosilicate on alumina catalysts and superior to commercial ZSM-5 aluminosilicate catalysts.

What is claimed is:

1. A boroaluminosilicate molecular sieve having an average crystallite size less than 2 μm.

2. The boroaluminosilicatc molecular sieve of claim 1, wherein the average crystallite size is between 50 nm to 1 μm.

3. The boroaluminosilicate molecular sieve of claim 1, wherein the alkali metal content is less than 400 ppmw.

4. The boroaluminosilicate molecular sieve of claim 1, wherein the alkali metal content is less than 150 ppmw.

5. A method of increasing the proportion of p-xylene (pX) in a hydrocarbon-containing feed stream comprising xylene isomers, said method comprising:
contacting the hydrocarbon-containing feed stream with an isomerization catalyst of claim 1 and under conditions suitable to yield a stream enriched in p-xylene with respect to the hydrocarbon-containing feed stream.

6. A method of increasing the proportion of p-xylene (pX) in a hydrocarbon-containing feed stream comprising xylene isomers, said method comprising:
contacting the hydrocarbon-containing feed stream with an isomerization catalyst under conditions suitable to yield a stream enriched in p-xylene with respect to the hydrocarbon-containing feed stream, wherein
the isomerization catalyst comprises a boroaluminosilicate molecular sieve prepared using an amine base.

7. The method of claim 6, wherein the boroaluminosilicate molecular sieve is prepared using ethylenediamine.

8. The method of claim 6, wherein the boroaluminosilicate molecular sieve has an alkali metal content is less than 400 ppmw.

9. The method of claim 6, further comprising recovering byproducts from the pX enriched stream.

10. The method of claim 6, wherein the byproducts contain 1.5 wt. % or less net toluene byproduct.

11. The method of claim 6, wherein the byproducts contain 3.5 wt. % or less net C₉-byproducts.

12. The method of claim 6, wherein the pX enriched stream contains less than 0.7 wt. % net trimethylbenzene byproduct.

13. The method of claim 6, wherein the pX enriched stream contains less than 1.0 wt. % net toluene.

14. The method of claim 6, wherein the pX enriched stream contains less than 0.5 wt. % net trimethylbenzene byproduct.

15. The method of claim 6, wherein the pX enriched stream contains at least 23.5 wt. % pX/X and less than 1.5 wt. % net toluene byproduct.

16. The method of claim 6, wherein the pX enriched stream contains at least 23.5 wt. % pX/X and less than 1.0 wt. % net trimethylbenzene byproduct.

17. The method of claim 6, wherein
the pX enriched stream contains at least 23.5 wt. % pX/X and a ratio of pX/X to the sum of net wt. % trimethylbenzene byproduct and net wt. % toluene byproduct of greater than 4.0.

18. The method of claim 6, wherein the hydrocarbon-containing feed stream comprises at least 80 wt. % xylene isomers and pX/X of less than 12 wt. %.

19. The method of claim 6, wherein the hydrocarbon-containing feed stream is contacted with the isomerization catalyst in the presence of hydrogen.

20. The method of claim 6, further comprising recovering a pX product from the pX enriched stream, thereby forming a pX-lean stream.

21. The method of claim 20, wherein the pX-lean stream is recycled for use as the hydrocarbon-containing feed stream.

22. The method of claim 6, further comprising forming a combination stream by combining a make-up feed stream comprising xylene isomers with the pX enriched stream.

23. The method of claim 21, further comprising recovering a pX product from the combination stream, thereby forming a pX-lean stream for use as a hydrocarbon-containing feed stream.

24. The method of claim 22, further comprising recovering byproducts from the combination stream.

25. The method of claim 6, further comprising contacting the hydrocarbon-containing feed stream with an ethylbenzene (EB) conversion catalyst under conditions suitable to reduce the EB content of the hydrocarbon-containing feed stream.

26. The method of claim 25, wherein the hydrocarbon-containing feed stream is contacted with the EB conversion catalyst prior to being contacted with the isomerization catalyst.

27. The method of claim 25; wherein the hydrocarbon-containing feed stream is contacted with the EB conversion catalyst and the isomerization catalyst in a single reaction zone.

28. The method of claim 25, wherein the EB conversion catalyst comprises an AI-MFI molecular sieve or a ZSM-5-type molecular sieve.

29. The method of claim 6, wherein the isomerization catalyst further comprises a support.

30. The method of claim 29, wherein the support comprises alumina, silica, titania, or a mixture thereof.

31. The method of claim 30, wherein the support comprises silica.

32. The method of claim 30, wherein the support comprises titania.

33. The method of claim 30, wherein the support comprises alumina.

34. The method of claim 33, wherein the support comprises a mixture of alumina and silica.

35. A catalyst system for enriching a mixed xylenes feed in p-xylene comprising
a first bed comprising an ethylbenzene (EB) conversion catalyst and a second bed comprising an isomerization catalyst that comprises a boroaluminosilicate molecular sieve.

36. The catalyst system of claim 35, wherein the boroaluminosilicate molecular sieve has an alkali metal content less than 400 ppmw.

37. The catalyst system of claim 35, wherein the boroaluminosilicate molecular sieve has an average crystallite size less than 2 μm.

38. The catalyst system of claim 35, wherein the boroaluminosilicate molecular sieve has an average crystallite size between 50 nm to 1 μm.

39. The catalyst system of claim 35, wherein the boroaluminosilicate molecular sieve is prepared using a base.

40. The catalyst system of claim 39, wherein the isomerization catalyst is prepared by:
combining a boron source, an aluminum source, a silica sol, and a template with the base to form a reaction mixture;
warming the reaction mixture to provide a product mixture comprising a solid;
isolating the solid from the product mixture; and
calcining the solid to yield the isomerization catalyst.

41. The catalyst system of claim 40, wherein the template is tetrapropylammonium bromide or tetrapropylammonium hydroxide.

42. The catalyst system of claim 39, wherein the base comprises ethylenediamine.

43. The catalyst system of claim 35, wherein the EB conversion catalyst comprises an AI-MFI molecular sieve or a ZSM-5-type molecular sieve.

44. The catalyst system of claim 35, wherein the isomerization catalyst further comprises a support.

45. The catalyst system of claim 44, wherein the support comprises alumina, silica, titania, or a mixture thereof.

46. The catalyst system of claim 45, wherein the support comprises silica.

47. The catalyst system of claim 45, wherein the support comprises titania.

48. The catalyst system of claim 45, wherein the support comprises alumina.

49. The catalyst system of claim 45, wherein the support comprises a mixture of alumina and silica.

50. The catalyst system of claim 35, wherein the first bed is disposed over the second bed.

51. The catalyst system of claim 50, wherein a guard bed comprising a hydrogenation catalyst component is disposed over the first bed.

52. The catalyst system of claim 50, wherein a guard bed comprising a hydrogenation catalyst component is disposed between the first bed and the second bed.

53. A xylene isomerization reactor comprising a reaction zone containing a catalyst system of claim 35.

54. The method of claim 1, wherein the isomerization catalyst further comprises a hydrogenation catalyst component.

55. The catalyst system of claim 35, further comprising a hydrogenation catalyst component.

56. The method of claim 1, wherein the isomerization catalyst has an aluminum content of 0.01 wt % to 3.3 wt %.

57. The method of claim 1, wherein the isomerization catalyst has a boron content of 0.01 wt % to 2.0 wt %.

58. The catalyst system of claim 35, wherein the isomerization catalyst has an aluminum content of 0.01 wt % to 3.3 wt %.

59. The catalyst system of claim 35, wherein the isomerization catalyst has a boron content of 0.01 wt % to 2.0 wt %.
